(19) United States
(12) Patent Application Publication
(10) Pub. No.: US 2006/0094893 A1
(43) Pub. Date: May 4, 2006

(54) Process for preparing cyclic carbonates

(76) Inventors: Darbha Srinivas, Maharashtra (IN); Rajendra Srivastava, Maharashtra (IN); Paul Ratnasamy, Maharashtra (IN)

Correspondence Address: OSTROLENK FABER GERB & SOFFEN, 1180 AVENUE OF THE AMERICAS, NEW YORK, NY, 100368403, US

(21) Appl. No.: 10/976,706

(22) Filed: Oct. 29, 2004

(51) Int. Cl. C07C 67/36

(52) U.S. Cl. 560/114

(57) ABSTRACT

The present invention relates to a process for the preparation of cyclic carbonates comprising contacting an epoxide with CO₂ in the presence of a titanosilicate catalyst and a base co-catalyst at a temperature above 313 K and a pressure above 2 bar for a period of 0.5 to 8 hrs and isolating the formed cyclic carbonate from the reaction mixture by conventional methods.

### FIELD OF THE INVENTION

The present invention relates to a process for preparing cyclic carbonates. More particularly, the present invention relates to an efficient, eco-friendly, clean process for preparing cyclic carbonates. Still more particularly the present invention relates to a process for preparing cyclic carbonates by contacting an epoxide with CO₂ in the presence a titanosilicate catalyst and a base co-catalyst and isolating formed cyclic carbonate from the reaction mixture.

### BACKGROUND OF THE INVENTION

Cyclic carbonates are important raw materials for engineering plastics such as polycarbonates. Currently, polycarbonates are manufactured using phosgene, a highly toxic, irritating and corrosive gas, inhalation of which causes fatal respiratory damage. Cyclic carbonates are also known for their application as organic solvents and octane booster. The total demand of polycarbonates is more than 1.5 million tons per annum. The demand for polycarbonates is expected to increase by approximately 9% per year. Plastics of this material are widely used in electric and electronic industry, building industry, optical data storage media, automotive industry, package industry, headlamp diffuser lense and bottles for water and milk. Polycarbonates of aliphatic type are used as plasticizers, stabilizers for vinyl chloride polymers, co-monomers in polyurethane synthesis, lubricants, elastomers (functionalized PC with pendent vinyl group) and biodegradable and biomedical materials for drug delivery.

Polycarbonates, for example bisphenol-A based aromatic polycarbonates, are commercially manufactured by condensation of 4-hydroxydiphenylbutane and phosgene (COCl₂) in the presence of substituted amines and alkali (Encyclopedia of Chemical Processing and Design, Vol 40, Ed. by J. J. McKetta and W. A. Cunningham, Marcel Dekker Inc., New York, 1992, p. 136 and Ulmann's encyclopedia of Industrial Chemistry, Vol. A 21, Ed. by B. Elvers, S. Hawkins and G. Schulz, 5^{th} ed. VCH Verlagsgesellschaft, mbH, Germany 1992, p. 207). This method of preparation employing phosgene is highly toxic and hazardous and therefore, eco-friendly routes for preparation, of polycarbonates are highly desirable. Preparation of polycarbonates from cyclic carbonates is an alternative attractive route.

Inoue et al. (J. Poly. Sci. Polym. Lett. Vol. 7, pp. 298 (1969)) reported that cyclic and polycarbonates can be prepared by the reaction of CO₂ and epoxides in the presence of organozinc catalyst, thereby opening a potentially benign route to polycarbonates using CO₂, a green house gas. Unfortunately, the metal complex catalysts that were found useful were also toxic, water and air-sensitive, caused handling problems, and in addition required high temperature and pressure for good conversion and selectivity. It was also subsequently found that this reaction takes place in the presence of a variety of complexes from simple alkali salts to classical organometallic complexes to different extents. Porphyrin (F. Kijima et al., J. Am. Chem. Soc. 108 (1986) 391; T. Aida et al Macromolecules 15 (1982) 682 and 19 (1986) 8), phthalocyanine (Ji et al., Appl. Catal. A: General 203 (2000) 329) and Schiff base (J. Am. Chem. Soc. 123 (2001) 11498) complexes are some of those homogeneous catalysts reported to catalyzed cycloaddition reaction. But high concentration of the catalyst (≧1 mol %) is required and necessitates expensive catalyst separation and product purification.

The following patents on cyclic carbonate preparation all employ homogeneous catalysts.

U.S. Pat. No. 4,824,969 Exxon Research & Engineering Co.) reports a process for preparing cyclic carbonate esters from olefins in a single reaction mixture using osmium compound, copper containing co-catalyst I (e.g., CuBr₂), co-catalyst II (e.g., pyridine) and water. U.S. Pat. Nos. 4,826,887 and 4,826,953 (Shell Oil Co.) report the process for the preparation of polycarbonates in the presence of catalytic amounts of a double metal cyanine complex and one or more salts composed of at least bivalent metal ions and metal-free anions having a solubility in water of at least 1 g/100 ml and one or more no-metal containing acids.

U.S. Pat. No. 6,469,193 reports the preparation of aliphatic carbonates from aliphatic alcohols, alkyl halides and carbon dioxide in the presence of cesium carbonate and tetrabutyl ammonium iodide. U.S. Pat. No. 6,407,264 reports a process involving the reaction of alkylene oxide with carbon dioxide in the presence of a catalyst system comprising of a metal halide and pyridine or pyridine derivative.

U.S. Pat Nos. 6,399,536, 5,391,767 and 6,288,202 and UK Pat Appl. GB 2352449 A1, PCT Int. Appl. WO 2000008088 A1, Ger. Offen. DE 19737547 A1 and Eur. Pat. Appl. EP 864361 A2 are all related to this process.

There are a few reports on the use of solid catalysts such as silica supported guanidine (Barbarini et al Tetrahedron Lett. 44 (2003) 2931) and MCM-supported phthalocyanine (Lu et al., J. Mol. Catal. A: Chemical 186 (2002) 33) for this reaction, however larger amounts catalyst and long reaction times (>15 h) are needed for high yield of cyclic carbonate. Recently, Srivastava et al (Catal. Lett. 89 (2003) 81) have reported the synthesis of cyclic carbonates from olefins using metal phthalocyanines encapsulated in zeolite-Y.

### OBJECTS OF THE INVENTION

It is therefore one of the objects of the present invention to provide an efficient, eco-friendly process for the preparation of cyclic carbonates in high yields.

Another object is to provide a process for the production of cyclic carbonates wherein use of toxic phosgene is eliminated.

Yet another object of the present invention is to prepare cyclic carbonates from epoxide by contacting an epoxide with CO₂ in the presence a titanosilicate catalyst and a base co-catalyst, at a temperature above 313 K, a pressure above 2 bar.

### SUMMARY OF THE INVENTION

Accordingly, the present invention provides a process for the production of cyclic carbonates comprising contacting an epoxide with CO₂ in the presence of a titanosilicate catalyst and a base co-catalyst and isolating cyclic carbonate so formed from the reaction mixture.

In one embodiment of the invention the cyclic carbonate is selected from the group consisting of ethylene carbonate, propylene carbonate, butylenes carbonate, chloropropylene carbonate, styrene carbonate and cyclohexene carbonate.

In another embodiment of the invention the epoxide is selected from the group consisting of ethylene oxide, propylene oxide, chloropropylene oxide, cyclohexene oxide, styrene oxide and butylene oxide.

In a further embodiment of the invention, the titanosilicate catalyst is selected from the group consisting of TS-1, TiMCM-41, Ti-beta and an amorphous titanosilicate of the formula x TiO₂.(1-x)SiO₂ where x lies between 0.0005 to 0.04.

In another embodiment of the invention, the co-catalyst is a Lewis base selected from the group consisting of pyridine, pyridine derivatives, alkyl phosphene, aryl phosphene, alkyl ammonium salts and phosphonium salts.

In another embodiment of the invention, the step of contacting is carried out in the presence of a solvent selected from the group consisting of chlorohydrocarbon, acetonitrile, acetone, N,N-dimenthyl formamide, pyridine, 1,4-dioxane and water, preferably dichloromethane.

In a further embodiment of the invention, the step of contacting is carried out at a temperature above 313 K, a pressure above 2 bar for a period of 0.5 to 8 hrs.

In yet another embodiment of the invention, the selectivity for the cyclic carbonate is greater than or equal to 80%.

### DETAILED DESCRIPTION OF THE INVENTION

The present invention provides an efficient process preparation of cyclic carbonates from epoxides using titanosilicate catalysts. The catalysts are easily separable by centrifugation or simple filtration and reusable. More importantly, the catalysts are highly efficient and only a small amount is needed to carryout the reaction. The process is also atom-efficient and reaction conditions like temperature and pressure are moderate. Atomic dispersion, tetrahedral framework substitution and the unusual crystal field imposed at the Ti site by the silica framework are some of the possible reasons for the efficient activity of titanosilicate catalysts used in the present invention.

In the investigations leading to the present invention, it was found that the titanosilicate catalyst is highly efficient and could be easily separated from the products and reused. Metal ion dispersion and framework substitution are the possible reasons for the activity enhancement. Prior art catalysts are not sufficiently active and need additional expenses for catalyst separation. An easily separable catalyst system e.g., the catalyst of the present invention is beneficial. Hence, the solid catalysts of the present invention are not only efficient but avoid the tedious process of catalyst recovery characteristic of the prior art processes and eliminate the presence of toxic elements like metal ions in the products and effluents. If the metal ions are allowed to be present in the product they are expected to modify the physical and chemical properties of the products. Hence, the present invention is environmentally more beneficial. The present invention does not involve the toxic phosgene reactants and hence, unlike the commercial process it is safer.

The present invention therefore provides an improved process for the preparation of cyclic carbonates by contacting an epoxide with CO₂ in the presence a titanosilicate catalyst and a base co-catalyst. The contacting is carried out optionally in the presence of a solvent and at a temperature above 313 K, a pressure above 2 bar for a period of 0.5 to 8 hrs. The formed cyclic carbonate is then isolated from the reaction mixture by conventional methods.

The cyclic carbonate obtained can be ethylene carbonate, propylene carbonate, butylenes carbonate, chloropropylene carbonate, styrene carbonate and cyclohexene carbonate. The epoxide used in the process of the invention is selected from ethylene oxide, propylene oxide, chloropropylene oxide, cyclohexene oxide, styrene oxide and butylene oxide. The titanosilicate catalyst used is TS-1, TiMCM-41, Ti-beta or amorphous titanosilicate having the formula
x TiO₂.(1-x)SiO₂

where x lies between 0.0005 to 0.04 and characterized by the features presented in Table 1.

TABLE 1

| Sl. | | |
|---|---|---|
| No. | Catalyst | Characteristic features |
| 1. | TS-1 | Si/Ti ratio = 36 |
| | | XRD: orthorhombic at 298K and monoclinic at 80K |
| | | FT-IR: A band at 960 cm⁻¹. |
| | | Diffuse reflectance UV-visible: a band at 206 nm |
| | | Magnetic property: Diamagnetic |
| | | Oxidation state of Ti = +4 |
| | | BET Surface area = about 400 m²/g |
| 2. | TiMCM-41 | Si/Ti ratio = 46 |
| | | XRD: M41S type characteristic reflections |
| | | FT-IR: A band at 950-960 cm⁻¹ |
| | | Diffuse reflectance UV-visible: a band at 220 nm |
| | | Magnetic property: Diamagnetic |
| | | Oxidation state of Ti = +4 |
| | | BET surface area = 963 m²/g |
| | | Average pore diameter = 3 nm |

The co-catalyst can be a Lewis base such as pyridine or pyridine derivatives, alkyl or aryl phosphene, alkyl ammonium salts and phosphonium salts. As explained above, the reaction can be carried out in the presence or absence of a solvent. The solvent when used is selected from chlorohydrocarbon, acetonitrile, acetone, N,N-dimenthyl formamide, pyridine, 1,4-dioxane or water, preferably dichloromethane.

The process of the present invention is phosgene-free and more environmental-friendly. The catalyst is a solid and the reaction takes place in a heterogeneous condition. The product (cyclic carbonate) obtained is a liquid and the solid catalyst can be easily separated from products by centrifugation/filtration and reused with little loss in activity. It is observed that the selectivity for the cyclic carbonate is greater than or equal to 80%.

The present invention is illustrated hereinbelow with examples, which are illustrative and should not be construed to limit the scope of the present invention in any manner.

### EXAMPLE 1

Microporous TS-1 used in the reaction was prepared according to the published procedure of Thangaraj et al J. Catal. 130, 1 (1991). To 45 g of tetraethylorthosilicate (TEOS), 50 g of tetrapropyl ammonium hydroxide (20% aq. TPAOH solution, Aldrich) was added. To the resultant liquid mixture 2.2 g of Ti butoxide in 10 g isopropyl alcohol was added drop-wise under vigorous stirring. The clear liquid obtained was stirred further for 15 more minutes. Then 20 g of TPAOH in 70 g double distilled water was added slowly to the above Score and the mixture then stirred at 348-353 K for about 3 hrs. Crystallization was carried out at 443 K for 1 day at static conditions. The solid obtained was filtered, washed and dried at 373 K for 5 h in static air. Si/Ti ratio of the catalyst=36 and specific surface area=400 m²/g.

### EXAMPLE 2

Mesoporous TiMCM-41 using in the reactions was prepared from the synthesis gel of the following molar composition (in terms of oxides); SiO₂:0.03TiO₂:0.089(CTMA)₂O:0.155(TMA)₂O:18H₂O.

Fumed silica (99%, Sigma), tetramethylammonium silicate (TMA silicate; 10 wt % silica solution, TMA/SiO₂=0.5; SACHEM, USA), cetyltritrimethylammonium chloride/hydroxide (CTMACl/OH; 17.9 wt % Cl and 6.7 wt % OH) and Ti butoxide (Aldrich) were used in the preparation. In a typical synthesis 24.6% solution of CTMACl/OH (16.7 g) was taken in a polypropylene beaker and 2.08 g TMAOH dissolved in 10 g water and 13.6 g TMA silicate were added to it while stirring. The thick gel formed was stirred for 15 min. Fumed silica (3.1 g) was then added slowly in about 10 min to the above mixture under stirring. The stirring was continued for 1 h after complete addition. To this thick slurry, 0.502 g of Ti butoxide (for Si/Ti=50) in 5-6 g of isopropanol was added. Stirring was continued for 1 hr. The pH of the final slurry was maintained at 11.5. The mixture was then transferred to a stainless steel autoclave and heated at 383 K for 5 days. The solid material (TiMCM-41) was filtered, washed with deionized water and dried at 373 K in air. The product was then calcined at 823 K, first, in flowing nitrogen (for 3 hrs) and then, in flowing air (for 6 hrs) to remove the organic material. Si/Ti ratio of the catalyst was found to be 46 by XRF, specific surface area=963 m²/g, pore volume=0.9 cm³ and average pore diameter=30 Å.

### EXAMPLE 3

This example illustrates the procedure for the preparation of chloropropylene carbonate from epichlorohydrin and carbon dioxide using TS-1 catalyst and N,N-dimethyl aminopyridine (DMAP) co-catalyst. In a typical reaction 18 mmol of epichlorohydrin, 100 mg of TS-1, 0.0072 mmol of DMAP and 20 ml of CH₂Cl₂ were taken in a 100 ml stainless steel pressure reactor. The reactor was pressurized to 100 psig with CO₂ and then the temperature was raised to 393 K. Reaction was conducted for 4 hrs. The reactor was then cooled to 298 K, unreacted CO₂ was vented out, catalyst was separated by filtration and the products were analyzed by GC (Varian 3400) and identified by GC-MS (Shimadzu QP-5000), IR (Perkin Elmer 2000) and ¹H NMR (Bruker AC 200).

### EXAMPLE 4

This example illustrates the procedure for the preparation of chloropropylene carbonate from epichlorohydrin and carbon dioxide using Ti-MCM-41 catalyst and N,N-dimethyl aminopyridine (DMAP) co-catalyst. In a typical reaction 18 mmol of epichlorohydrin, 100 mg of Ti-MCM41, 0.0072 mmol of DMAP and 20 ml of CH₂Cl₂ were taken in a 100 ml stainless steel pressure reactor. The reactor was pressurized to 100 psig with CO₂ and then the temperature was raised to 393 K. Reaction was conducted for 4 hrs. The reactor was then cooled to 298 K, unreacted CO₂ was vented out, catalyst was separated by filtration and the products were analyzed by GC (Varian 3400) and identified by GC-MS (Shimadzu QP-5000), IR (Perkin Elmer 2000) and ¹H NMR (Bruker AC 200).

### EXAMPLE 5

This example illustrates the procedure for the preparation of propylene carbonate from propylene oxide and carbon dioxide using TS-1 catalyst and N,N-dimethyl aminopyridine (DMAP) co-catalyst. In a typical reaction 18 mmol of propylene oxide, 100 mg of TS-1, 0.0072 mmol of DMAP and 20 ml of CH₂Cl₂ were taken in a 100 ml stainless steel pressure reactor. The reactor was pressurized to 100 psig with CO₂ and then the temperature was raised to 393 K, Reaction was conducted for 6 hrs. The reactor was then cooled to 298 K, unreacted CO₂ was vented out, catalyst was separated by filtration and the products were analyzed by GC (Varian 3400) and identified by GC-MS (Shimadzu QP-5000), IR (Perkin Elmer 2000) and ¹H NMR (Bruker AC 200).

### EXAMPLE 6

This example illustrates the procedure for the preparation of styrene carbonate from styrene oxide and carbon dioxide using TS-1 catalyst and N,N-dimethyl aminopyridine (DMAP) co-catalyst. In a typical reaction 18 mmol of styrene oxide, 100 mg of TS-1, 0.0072 mmol of DMAP and 20 ml of CH₂Cl₂ were taken in a 100 ml stainless steel pressure reactor. The reactor was pressurized to 100 psig with CO₂ and then the temperature was raised to 393 K. Reaction was conducted for 8 hrs. The reactor was then cooled to 298 K, unreacted CO₂ was vented out catalyst was separated by filtration and the products were analyzed by GC (Varian 3400) and identified by GC-MS (Shimadzu QP-5000), IR (Perkin Elmer 2000) and ¹H NMR (Bruker AC 200).

### EXAMPLE 7

This example illustrates the procedure for the preparation of styrene carbonate from styrene oxide and carbon dioxide using Ti-MCM-41 catalyst and N,N-dimethyl aminopyridine (DMAP) co-catalyst. In a typical reaction 18 mmol of styrene oxide, 100 mg of Ti-MCM-41, 0.0072 mmol of DMAP and 20 ml of CH₂Cl₂ were taken in a 100 ml stainless steel pressure reactor. The reactor was pressurized to 100 psig with CO₂ and then the temperature was raised to 413 K. Reaction was conducted for 10 hrs. The reactor was then cooled to 298 K, unreacted CO₂ was vented out, catalyst was separated by filtration and the products were analyzed by GC (Varian 3400) and identified by GC-MS (Shimadzu QP-5000), IR (Perkin Elmer 2000) and ¹H NMR (Bruker AC 200).

### EXAMPLE 8

This example illustrates the procedure for the preparation of 1-butene carbonate from 1-butene oxide and carbon dioxide using TS-1 catalyst and N,N-dimethyl aminopyridine (DMAP) co-catalyst. In a typical reaction 18 mmol of 1-butene oxide, 100 mg of TS-1, 0.0072 mmol of DMAP and 20 ml of CH₂Cl₂ were taken in a 100 ml stainless steel pressure reactor. The reactor was pressurized to 100 psig with CO₂ and then the temperature was raised to 393 K. Reaction was conducted for 6 hrs. The reactor was then cooled to 298 K, unreacted CO₂ was vented out, catalyst was separated by filtration and the products were analyzed by GC (Varian 3400) and identified by GC-MS (Shimadzu QP-5000), IR (Perkin Elmer 2000) and ¹H NMR (Bruker AC 200).

The catalytic activity data of titanosilicate catalysts are listed in TABLE 2. Spectral characteristics of the product cyclic carbonate are as follows:

Chloropropylene carbonate—IR(cm⁻¹): ν_{c-o}, 1800, ν_{c-o}, 1133, 1080; ¹H NMR (CDCl₃), δ(ppm): 5.03-4.94 (1H, m), 4.61-4.52 (1H, q), 4.44-4.35 (1H, q), 3.84-3.74 (2H, m).

Propylene carbonate—IR(cm⁻¹): ν_{c-o}, 1793, ν_{c-o}, 1121, 1078; ¹H NM (CDCl₃), δ(ppm): 4.88-4.77 (1H, m), 4.55-4.49 (1H, t), 4.01-3.96 (1H, t), 1.45 (3H, d).

TABLE 2
Synthesis of cyclic carbonates over titanosilicate catalysts

| | | | | Selectivity |
|---|---|---|---|---|
| | | | | for cyclic |
| | | | Conversion | carbonate |
| Example | Catalyst | Epoxide | (mol %) | (mol %) |
| 3 | TS-1 | Epichlorohydrin | 85.4 | 92.6 |
| 4 | TiMCM-41 | Epichlorohydrin | 78.8 | 84.0 |
| 5 | TS-1 | Propylene oxide | 66.8 | 84.6 |
| 6 | TS-1 | Styrene oxide | 44.7 | 45.5 |
| 7 | TiMCM-41 | Styrene oxide | 98.1 | 73.1 |
| 8 | TS-1 | 1-butene oxide | 76.6 | 70.9 |

The process described above has the combined unique advantages of high epoxide conversion accompanied with high selectivity for cyclic carbonate. The process is eco-friendly and does not involve toxic reactants like phosgene. Little effort is required to separate the catalyst. The separated catalysts can be reused with no significant loss in activity. The catalysts of the present invention are highly efficient for the preparation of cyclic carbonates from epoxides.

We claim:

1. A process for the production of cyclic carbonates comprising contacting an epoxide with CO₂ in the presence of a titanosilicate catalyst and a base co-catalyst and isolating cyclic carbonate so formed from the reaction mixture.

2. A process as claimed in claim 1 wherein the cyclic carbonate formed is selected from the group consisting of ethylene carbonate, propylene carbonate, butylenes carbonate, chloropropylene carbonate, styrene carbonate and cyclohexene carbonate.

3. A process as claimed in claim 1 wherein the epoxide is selected from the group consisting of ethylene oxide, propylene oxide, chloropropylene oxide, cyclohexene oxide, styrene oxide and butylene oxide.

4. A process as claimed in claim 1 wherein the titanosilicate catalyst is selected from the group consisting of TS-1, TiMCM-41, Ti-beta and an amorphous titanosilicate of the formula x TiO₂.(1-x)SiO₂ where x lies between 0.0005 to 0.04.

5. A process as claimed in claim 1 wherein the co-catalyst is a Lewis base selected from the group consisting of pyridine, pyridine derivatives, alkyl phosphene, aryl phosphene, alkyl ammonium salts and phosphonium salts.

6. A process as claimed in claim 1 wherein the step of contacting is carried out in the presence of a solvent selected from the group consisting of chlorohydrocarbon, acetonitrile, acetone, N,N-dimenthyl formamide, pyridine, 1,4-dioxane and water.

7. A process as claimed in claim 6 wherein the solvent is dichloromethane.

8. A process as claimed in claim 1 wherein the step of contacting is carried out at a temperature above 313 K, a pressure above 2 bar for a period of 0.5 to 8 hrs.

9. A process as claimed in claim 1 wherein the selectivity for the cyclic carbonate is greater than or equal to 80%.
